(19) United States
(12) Patent Application Publication
(10) Pub. No.: US 2024/0285965 A1
(43) Pub. Date: Aug. 29, 2024

(54) LIGHT EMITTING UNIT BASED ON LED

(71) Applicant: FARMER-LIGHTHOLDING A/S, Herning (DK)

(72) Inventor: POVL KAAS, Herning (DK)

(21) Appl. No.: 18/569,695

(22) PCT Filed: Jun. 21, 2022
(86) PCT No.: PCT/EP22/66814

§ 371 Date: Dec. 13, 2023

(30) Foreign Application Priority Data
Jun. 21, 2021 (EP) 21180593.2
Feb. 17, 2022 (EP) 22157172.2
Feb. 28, 2022 (EP) 22159226.4

(51) Int. Cl. A61N 5/06

(52) U.S. Cl. CPC A61N 5/0616; A61N 5/0624; A61N 2005/0628; A61N 2005/0652; A61N 2005/0661; A61N 2005/0663

(57) ABSTRACT

Methods, systems and light emitting units with light emitting diodes may be for facilitating production of vitamin D, reducing microbial pressure and optionally providing work light inside buildings. A light emitting unit is provided for reducing the microbial pressure in a building preferably in combination with sensors, and for promoting formation of natural vitamin D3 in animals and/or humans, and preferably also providing visible work light. A light emitting unit may include at least a first UV-B LED configured for emitting monochromatic UV-B light with maximum intensity between 292-302 nm, 295-299 nm, 296-298 nm, or 297 nm, and at least a second UV-B LED configured for emitting monochromatic UV-B light with maximum intensity between 278-288 nm, 281-285 nm, 282-284 nm, or 283 nm and/or at least a first UV-C LED configured for emitting monochromatic UV-C light with maximum intensity between 228-238 nm, 231-235 nm, 232-234 nm, or 233 nm.

### CROSS-REFERENCE TO RELATED APPLICATIONS

This application is the U.S. National Stage of PCT/EP2022/066814 filed on Jun. 21, 2022, which claims priority to European Patent Application 21180593.2 filed on Jun. 21, 2021, European Patent Application 22157172.2 filed on Feb. 17, 2022, and European Patent Application 22159226.4 filed on Feb. 28, 2022, the entire content of all are incorporated herein by reference in their entirety.

### FIELD OF THE INVENTION

The present disclosure relates to methods, systems and light emitting units based on light emitting diodes, in particular for increasing and/or facilitating the production of vitamin D, reducing the microbial pressure and optionally providing work light inside buildings such as animal farm production facilities, hospitals, offices, premises, storage facilities, manufacturing facilities, grocery stores, schools/classrooms, etc. A light emitting unit is provided for reducing the microbial pressure in a building, in particular from zoonoses, aerosols, bacteria and virus on surfaces of the animals and/or humans and surfaces of the interior of the building, while at the same time being configured for promoting the formation of natural vitamin D3 in animals and/or humans, and preferably also providing visible work light.

### BACKGROUND OF THE INVENTION

In animal farming, animals such as pigs, piglets, cattle or other domesticated animals are kept inside in animal housing facilities. The conditions in animal housing facilities may promote the growth of a wide diversity of microorganisms including bacteria, such as methicillin resistant Staphylococcus aureus (MRSA), and virus, such as swine acute diarrhea syndrome-coronavirus (SADS-COV). Presence of airborne microorganisms in animal housing facilities affects the quality of air in those facilities leading to exposing animals, workers of the facilities as well as people in neighbouring areas to pathogens. Intensive farming may result in an unsatisfactory increased level of microbial pressure, in particular from zoonoses and aerosols, inside the animal farm production facilities, potentially leading to significant health risks.

A variety of measures are typically taken to control the microbial pressure inside the animal housing facilities in order to ensure a good air quality and the well-being of the animals kept in the animal housing as well as the well-being of the employees/workers in the farm, for example admission to the animals is controlled and restricted, thereby decreasing the risk of contamination. However, the spread of infectious diseases between animals and between animals and humans, such as zoonoses, still poses a significant problem in animal housing environments.

During the corona pandemic the microbial pressure inside any type of building, such as hospitals, offices, premises, manufacturing facilities, grocery stores, schools/classrooms, etc., has also been a subject of interest, in particular to protect humans spending hours inside buildings from infectious diseases, like SARS-COVID-19.

This is further highlighted by the fact that humans and domesticated animals in many cases are not able to naturally produce, at a satisfactory level, vitamin D3. Natural light (sunlight) enhances the natural production of vitamin D3 in the skin of humans and animals. Vitamin D3 is produced in the skin from 7-dehydrocholesterol by ultra violet (UV) light of the B-type (UV-B). UV-B is present in the spectrum of natural light and hence the exposure of skin to natural light drives the formation of Natural vitamin D3 (ND3) in the skin. ND3 has a crucial function in the immune system and in the development and maintenance of the skeleton and bones.

With humans and domestic animals spending a large fraction of the day indoors, they will in many cases receive insufficient doses of UV-B for formation of a sufficient level of ND3. As a result, vitamin D3 deficiencies are not uncommon in animal farm housing environments and in humans living in the Northern hemisphere, with significant health implications relates thereto. One typical health effect of low ND3 levels is a weakened immune system, which in combination with a poor air quality makes them prone to infections.

In order to ensure humans and animals getting a sufficient amount of ND3 various approaches have been used. One approach for animals is to feed the animal a synthetic vitamin D3 (SD3) in the form of dietary supplement added to the animal feed. One example is the enriching of animal food with SD3 or by adding a pill or a powder of SD3. And dietary vitamin SD3 supplements for humans are also used globally.

Even though SD3 and ND3 are chemically identical they function differently in the body of animals and humans. SD3 does not bind to the proper transport proteins, in the manner that ND3 does, but instead SD3 remains in the residual fat of the blood after it has been absorbed through the intestinal tract. This is considered crucial for the biological effect of the vitamin and explains why large doses of SD3 are toxic whereas ND3 cannot be overdosed.

Animals or humans who have been infected by a bacterial disease can be treated with antibiotics. Although, for most diseases this cures the animal or human, it is nevertheless unsatisfactory not only from a health point of view, but because such antibiotic treatment is expensive and causes economic losses for the farmers. Furthermore, there is a constant increase in antibiotic resistant species, including the aforementioned MRSA, which have emerged as a major concern for the well-being of a wide range of domestic animal species, with serious economic consequences to the farmers as a result.

Consequently, lighting conditions inside buildings may have significant implications on the well-being and health of humans and/or animals spending hours inside. On top of that lighting conditions from visual light inside buildings are important for the visual perception of objects, as perceived by the cones and rods of the human eye, and are formed based on for example the lighting level, the spatial distribution and the colour rendering. Poor lighting conditions in for example a school or a work environment may lead to eye-strain, fatigue, headaches and stress. This in turn leads to a higher risk for accidents, a lowered productivity, and a lowered quality of life in general.

### SUMMARY OF THE INVENTION

To optimize the lighting conditions in a building it is a purpose of the present disclosure to provide a light emitting unit, a system and a method for reducing the microbial pressure inside a building, in particular in specific rooms in buildings, facilitating the formation of ND3, and preferably also to provide ideal working light conditions. The light emitting units, devices, systems, and methods disclosed herein may in certain aspects benefit from the disclosure of PCT/EP2020/067069 by the same inventor, which is therefore incorporated by reference in its entirety.

It is a purpose of the present disclosure to provide a light emitting unit that is configured to emit light in a number of wavelength ranges, e.g., two, three or more important wavelength ranges, particularly in the UV-B range, optionally also in the UV-C range and optionally also in the visible light range. The present light emitting unit is aimed at replacing for example prior art lamps that provide visible working light in buildings for humans and in farm production facilities. In case of light emitting units for farm production facilities with the further functionality of 1) providing visible light in a wavelength range and/or colour temperature that does not disturb the animals, and 2) providing light in one or more predefined wavelength ranges that help to reduce the microbial pressure inside the farm production facility. In that regard it is an advantage if the present light emitting unit is provided as only a single lamp, e.g., on a single (printed) circuit board. The present disclosure may therefore relate to a light emitting unit having a single lamp that is configured to emit light, at unusual high energies, in two, three or more important wavelength ranges, including the UV-B range and optionally UV-C range and the visible light range. A single lamp with LED Chips allows for significant cost reductions, both in terms of installation costs and running costs.

An embodiment of the present disclosure therefore relates to a light emitting unit for 1) reducing the microbial pressure, and 2) stimulating the production of natural vitamin D3, the light emitting unit comprising at least one UV-B Light Emitting Diode (LED) configured for emitting monochromatic UV-B light, and/or at least one UV-C Light Emitting Diode (LED) configured for emitting monochromatic UV-C light.

A preferred embodiment relates to a light emitting unit for 1) reducing the microbial pressure, and 2) stimulating the production of natural vitamin D3, the light emitting unit comprising at least a first UV-B LED configured for emitting monochromatic UV-B light having a maximum intensity between 292-302 nm, preferably between 295-299 nm, more preferably between 296-298 nm, most preferably at 297 nm, and
at least a second UV-B LED configured for emitting monochromatic UV-B light having a maximum intensity between 278-288 nm, preferably between 281-285 nm, more preferably between 282-284 nm, most preferably at 283 nm and/or
at least a first UV-C LED configured for emitting monochromatic UV-C light having a maximum intensity between 228-238 nm, preferably between 231-235 nm, more preferably between 232-234 nm, most preferably at 233 nm.

This embodiment is preferred because the 297 nm light provides for stimulation of the production of natural vitamin D3 in both humans and animals, the 283 nm light provides for reduction of the microbial pressure in animal farm productions facilities, and the 233 nm light provides for reduction in the microbial pressure in buildings housing humans, e.g. hospitals, schools, etc. However, possibly all these wavelengths can be provided in a single lamp, possibly on a single circuit board, and then the control of the individual LED chips are provided by software, such that suitable wavelengths are selected for the purpose and/or location of the lamp.

In particular it has been found advantageous to combine two UV-B wavelengths separated by 10-20 nm and selected from of each part of the UV-B spectrum, i.e., a low wavelength UV-B LED close to the UV-C spectral range and a higher UV-B LED closer to the UV-A spectral range, e.g., 275-285 or 278-288 nm in combination with 292-302 nm provided from different LEDs, because the combination of such two UV-B LEDs can both provide reduction of the microbial pressure and stimulation of ND3 production. Additional reduction of the microbial pressure can be provided by adding one or more UV-C wavelengths, e.g., at least one UV-C LED configured for emitting monochromatic UV-C light, preferably with wavelengths in a range of 215-240 nm, e.g., 222 nm, 230 nm, 233 nm and/or 260 nm.

Another particularly advantageous embodiment is to combine a UV-B wavelength from the upper end of the UV-B spectrum, e.g. 297±5 nm, with an UV-C wavelength around 230±10 nm or 230±5 nm, or 233±5 nm such as 233 nm, because the combination of such wavelengths can both provide reduction of the microbial pressure from the non-harmful 233 nm UV-C light, and stimulation of ND3 production from the UV-B light.

This light emitting unit may in particular be configured for reducing the microbial pressure in an animal farm production facility. The light emitting unit may comprise a plurality of LED light sources. The light emitting unit may advantageously be configured for emitting polychromatic/broadband visible light with wavelengths in the range of 380 nm-750 nm, and 2) not emitting (broadband) light below 285 nm, preferably below 270 nm, except emittance of monochromatic light at one or more predefined wavelengths, such as 222 nm, 230 nm, 233 nm and/or 260 nm. The light emitting unit may furthermore advantageously be configured for emitting monochromatic light at one or more additional predefined wavelengths, for example 295 nm or 297 nm. In particular it is important that the selected wavelengths are not harmful to humans or animals, and 230±10 nm and 295±10/297±5 nm are examples of such non-harmful wavelength ranges.

In an embodiment of the present disclosure, the light emitting unit is configured to emit polychromatic visible light and monochromatic light in wavelength ranges and energies selected for the inactivation of microorganisms, for the production of ND3 and for the provision of visible work light. At the same time, the light emitting unit may be configured to emit a low amount of energy, or even zero energy, having a wavelength below the UV-B range, however possibly except for monochromatic light at preselected wavelengths. It is a further preference that the light emitting unit is configured such that it can be received by a single standard lamp socket.

Good working light conditions is crucial for the wellbeing, safety and efficiency of humans staying inside for many hours per day, e.g., children in schools, patients and medical personnel in hospitals, and workers in storage facilities, manufacturing facilities, office facilities and animal farm facilities. The lighting conditions are important for the visual perception of objects, for example due to the lighting level, the spatial distribution and the colour rendering. The, by a human, perceived contrast of an object is for example a function of the absorption properties of the object, the intensity and spectral components of the lighting, and the sensitivity of the photoreceptor cells (i.e., cones, rods and intrinsically photosensitive retinal ganglion cells) in the retina. The presently disclosed light emitting unit may therefore be configured to provide visible light with a selected colour temperature, e.g., 2700 K is suitable for hospitals whereas 4500 K is suitable for farm production facilities.

### BRIEF DESCRIPTION OF DRAWINGS

FIGS. 1-2 show one embodiment of the presently disclosed light emitting unit based on LED technology with a circular housing and suitable for ceiling mounting;

FIGS. 3A-C show one embodiment of the presently disclosed light emitting unit based on LED technology with a rectangular housing and suitable for ceiling mounting;

FIG. 4 shows a test setup for illumination of S. aureus on glass beads with UV radiation using an embodiment of the presently disclosed light emitting unit;

FIGS. 5A-F show various combinations of UV-B, UV-C and white light LED chips on a common printed circuit board for use in the presently disclosed light emitting unit;

FIGS. 6A-F show various combinations of UV-B, UV-C and white light LED chips on a common printed circuit board for use in the presently disclosed light emitting unit, in particular suitable for use in human facilities, such as hospitals, schools, etc;

FIGS. 7A-E show various combinations of UV-B, UV-C, and white light LED chips on a common printed circuit board;

FIG. 8 shows a perspective illustrative view of a dual in-line package LED chip;

FIG. 9 shows an exemplary spectral distribution from an UV-B LED with a peak wavelength of 295 nm, a FWHM of 14 nm and providing UVB light from approx. 275 to 315 nm;

FIG. 10A shows an exemplary spectral distribution from an UV-B LED with a peak wavelength of 275 nm, a FWHM of 15 nm providing UVB light from approx. 260 to 300 nm;

FIG. 10B shows an exemplary spectral distribution from an UV-B LED with a peak wavelength of 285 nm, a FWHM of 25 nm providing UVB light from approx. 265 to 315 nm;

FIG. 10C shows exemplary spectral distributions from two UV-B LEDs with a peak wavelengths of 280 and 295 nm (both with FWHM of 15 nm), respectively and their combined spectral distribution UVB light from approx. 260 to 320 nm;

FIGS. 11A-B show one embodiment of the presently disclosed system comprising a plurality of light emitting units installed near the ceiling of a hospital ward and with light sensors mounted on the wall for providing working light to the hospital ward and for illuminating the patients with UV light;

FIGS. 12A-B show one embodiment of the presently disclosed system comprising a plurality of light emitting units installed near the ceiling of a pig house for providing working light to the premises and for illuminating the pigs with UV light;

FIG. 13 is an illustration of glass beads in a petri dish used for measurement of bacteria removal in Example 1;

FIG. 14 is a graph showing the efficiency in removal of bacteria vs. the wavelength of the illumination light; and

FIGS. 15-17 illustrate removal of S. aureus inoculated on glass beads at different doses of UV irradiation with different UV-B wavelength setups: FIGS. 15A-B having 285±295 nm LEDs, FIGS. 16A-B having 280±297 nm LEDs and FIGS. 17A-B having 285 nm broad spectral LED.

### DETAILED DESCRIPTION OF THE INVENTION

As stated above the present disclosure relates to a light emitting unit for 1) reducing the microbial pressure, and 2) stimulating the production of natural vitamin D3, the light emitting unit comprising at least one UV-B Light Emitting Diode (LED) configured for emitting monochromatic UV-B light.

In one embodiment the light emitting unit comprises at least a second UV-B LED configured for emitting monochromatic UV-B light having a maximum intensity between 280-290 nm, more preferably between 283-287 nm, most preferably at 283 nm or 285 nm. This monochromatic UV-B LED light preferably has a full width half max (FWHM) spectral bandwidth of less than or equal to 50 nm, more preferably less than or equal to 40 nm, even more preferably less than or equal to 30 nm, most preferably less than or equal to 20 nm. Such an embodiment with a broad spectral single UV-B LED is preferred for use in animal farm production facilities because a large part of the UV-B spectrum is utilized in a cost-effective solution where it is not necessary to adjust the power ratio between different wavelengths in the UV-B spectrum.

In one embodiment the light emitting unit comprises at least a second UV-B LED configured for emitting monochromatic UV-B light having a maximum intensity between 275 nm-290 nm more preferably between 278 nm-288 nm, most preferably at 283 nm. Additionally, or alternatively at least a first UV-B LED configured for emitting monochromatic UV-B light having a maximum intensity between 290 nm-305 nm, more preferably between 292 nm-302 nm, most preferably at 297 nm. In one embodiment the monochromatic UV-B LED light has a full width half max (FWHM) spectral bandwidth of less than or equal to 25 nm, more preferably less than or equal to 20 nm, even more preferably less than or equal to 15 nm, most preferably less than or equal to 10 nm. In particular for the short UV-B wavelength LED, i.e., at around 283 nm, it is preferred to have a narrow spectral bandwidth of 15 nm or smaller, to reduce the amount of UV-C light, whereas the higher wavelength UV-B LED advantageously can have larger spectral bandwidth of around 15-20 nm. A combination solution with two different UV-B wavelengths at 283±5 nm and 297±5 nm is preferred for solutions where humans are involved because with such a combination solution it is possible to adjust the resulting spectrum in the UV-B range by adjusting the power of the different wavelength LED chips. And different spectra may be preferred for different applications, be it health or sterilization, or other.

In a further preferred embodiment, the light emitting unit comprises at least one UV-C LED configured for emitting monochromatic UV-C light, preferably configured for emitting monochromatic UV-C light with wavelengths in a range of 215-240 nm. For example, at least a first UV-C LED configured for emitting monochromatic UV-C light having a maximum intensity between 228-238 nm, preferably between 231-235 nm, most preferably at 233 nm. Additionally, or alternatively at least at least a second UV-C LED configured for emitting monochromatic UV-C light having a maximum intensity between 217-227 nm, preferably between 220-224 nm, most preferably at 222 nm.

Additionally, or alternatively at least a third UV-C LED configured for emitting monochromatic UV-C light having a maximum intensity between 255-265 nm, preferably between 258-262 nm, more preferably between 259-261 nm, most preferably at 260 nm. These three central wavelengths have been shown to be very efficient for reducing the microbial pressure, either used separately or two or three in combination.

As also stated above the combination of UV-C LED light at 233±5 nm and UV-B light at 297±5 nm has turned out to be particular advantageous due to the non-harmful UV-C light around 230 nm that is very efficient in terms of disinfection and the UV-B light around 300 nm that is also disinfective but also provides stimulation of ND3 production.

In one embodiment the monochromatic the monochromatic UV-C LED light has a full width half max (FWHM) spectral bandwidth of less than or equal to 25 nm, more preferably less than or equal to 20 nm, even more less than or equal to 15 nm, most preferably less than or equal to 10 nm. In particular for the short UV-C wavelength LED, i.e., at 222 nm, it is preferred to have a narrow spectral bandwidth of 5 nm or smaller, to reduce the amount of short wavelength UV-C light, whereas the higher wavelength UV-C LED advantageously can have larger spectral bandwidth of around 10-15 nm.

With LED technology several LED chips can be added to a common circuit board, i.e., in particular one or more visible light LED chips can be added such that the presently disclosed light emitting unit can be used for providing working light for humans in the building. I.e., in a further embodiment the presently disclosed light emitting unit comprises at least one visible light LED configured for emitting polychromatic visible light, preferably with wavelengths in the range of 380 nm-750 nm. The colour temperature of the visible light can be application dependent, i.e., for hospitals the colour temperature should preferentially be blue-shifted, preferably around 2700 K, whereas for animal farm facilities it has turned out the red-shifted visible light is an advantage, preferably around 4500 K. Hence, the presently disclosed light emitting unit may be configured such that the colour temperature of the visible polychromatic light is between 2500 K and 5000 K, such as around 4500 K, preferably around 2700 K.

The presently disclosed light emitting unit may be configured for not emitting light below 275 or even below 265 nm, except emittance of monochromatic UV-C light in one or more selected wavelengths, e.g., at 222 nm, 233 nm and/or at 260 nm.

The presently disclosed light emitting unit may be configured for emittance of the polychromatic visible light for a first predefined period of time of less than 16 hours per day, and configured for emittance of the monochromatic non-visible UV-B and/or UV-C light.

The inventor has realized that when rooms and equipment of e.g., premises are illuminated with wavelengths 233 nm, 283 nm, and/or 297 nm and optionally 222 nm, and/or 260 nm, the infection pressure will be reduced significantly in the room. The combination of these wavelengths eliminates significantly more viruses and bacteria than, for example, UV-C light alone. All disease-related bacteria and viruses that are based on aerosols will be reduced completely overnight in the illuminated room and all illuminated surfaces will be sterile.

Recently it has been shown that UV-C light around 230 nm, such as 230±10 nm, more preferably 230±5 nm, for example 233 nm, has very high bactericidal efficacy and skin tolerability. Tests have been conducted on an UV-C LED source with a central wavelength of 233 nm and FWHM of approx. 12 nm. The bactericidal efficacy was qualitatively analyzed using blood agar test and germ carrier tests using various MRSA strains and S. epidermidis with various soil loads. The compatibility of the germicidal radiation doses on excised human skin and reconstructed human epidermis was also analyzed. Cell viability, DNA damage and production of radicals were assessed in comparison to typical UV-C radiation from HED discharge lamps (222 nm, 254 nm) and UV-B radiation for clinical assessment. At a dose of 40 mJ/cm2, the 233 nm UV-C light source reduced the viable microorganisms by a log 10 reduction (LR) of 5 log 10 levels if no soil load was present. Mucin and protein containing soil loads diminished the effect to an LR of 1.5-3.3. A salt solution representing artificial sweat had only small effects on the reduction. The viability of the skin models was not reduced and the DNA damage was far below the damage evoked by 0.1 UVB minimal erythema dose, which can be regarded as safe. Furthermore, the induced damage vanished after 24 h. Irradiation on four consecutive days also did not evoke DNA damage. The radical formation was far lower than 20 min outdoor visible light would cause, which is classified as low radical load and can be compensated by the antioxidant defense system. From these tests it can be concluded that UV-C LEDs around 230 nm, such as 233 nm, can be used in combination with humans and animals with very low risk of causing harm, i.e. such UV-C LED can be used in light emitting units for reducing the microbial pressure in premises—and they can be used in combination with humans and animals, even when they are present in the premises, i.e. such UV-C LEDs can be turned on 24/7.

When a room is in use, for example, a school classroom or an office, the microbial pressure will be significantly reduced when the presently disclosed light emitting unit is switched on, and infection transmission from human to human will be significantly minimized. It would almost be impossible for there to be a human-to-human infection via the aerosols or from the areas that are illuminated.

During the day, the presently disclosed light emitting unit should only be used to illuminate with wavelengths of 233 nm, 283 nm and/or 297 nm (or 285 nm), while additional UV-C light, e.g. at 222 nm and/or 260 nm can also be added at night. It is not always necessary to illuminate with UV-C, as the combination of 283 nm and 297 nm is usually sufficient. FIG. 14 is a graph showing the efficiency in removal of the bacteria salmonella and E. coli vs. the wavelength of the illuminated light. As seen from the graph in FIG. 14 wavelengths below 285 nm are very efficient, whereas wavelengths above 290 nm are less efficient. However, wavelengths below approx. 275 nm are harmful to humans, with wavelengths around 233 nm as an exception. The surprising advantage of the presently disclosed combination of non-harmful invisible UV-B wavelengths is that 283 nm light degrades proteins, which are the protective surface of the virus, and the 297 nm light penetrates a thin organic surface, which for example 222 nm does not. Bacteria, and proteins that lie on top of each other, form a weak thin organic surface. This combination of 283 nm and 297 nm inactivates bacteria and viruses without harming humans while increasing the vitamin ND3 in the plasma of the illuminated persons. By increasing ND3 in the plasma, the immune system is significantly increased in humans, so that the infection with disease-causing viruses and bacteria is minimized and thus a significant reduction in the possibility of becoming infected and ill. Hence, even though FIG. 14 indicates that 290 nm light and above, e.g. 297±5 nm, is less efficient for removing bacteria is has been shown to be surprisingly effective in combination with lower wavelength UV-B light.

In a practical combination of UV-B wavelengths, 283 nm and 297 nm, the absolute energy/intensity and the relative energy/intensity of the wavelength are also important, and in the preferred embodiment of the presently disclosed light emitting unit, this can be varied. For example, in the case illumination of an animal farm facility with piglets a combination of energies in the ratio of four times 283 nm to eight times 297 nm. In the case of a hospital bed, e.g., on an intensive corona section, the relative amount of the low wavelength light should be increased, e.g., to an energy of ten times 283 nm compared to two times 297 nm light.

The presently disclosed light emitting unit may include one or more UV-C LEDs. When cleaning and thus using extra strong reduction of the microbial pressure in the room, UV-C light can be switched on e.g., 222 nm and/or 260 nm light, which is harmful to humans, in particular 260 m, but also very effective in reducing the microbial pressure. The UV-C light can be used alone or in combination with the UV-B light.

In the preferred embodiment of the presently disclosed light emitting unit all LEDs are mounted on a common circuit board, preferably a replaceable circuit board, such that only the circuit board needs to be replaced in case of service or malfunction.

The presently disclosed light emitting unit may comprise a housing, preferably metallic, preferably having cooling fins, the housing accommodating all LEDs of the light emitting unit.

### UV

In the present disclosure the term UV-A refers to the range from 315 to 400 nm, UV-B refers to the range of 280-315 nm, and UV-C refers to the range of 100-280 nm. The term Far UV (FUV) refers to the wavelength range 122-200 nm.

In various embodiments of the present disclosure a short wavelength UV-B LED is used centred around 283 nm, e.g. 278-288 nm or 285 nm, e.g. 283-290 nm. This type is referred to herein as an UV-B light source even though it is just around the lower limit of the UV-B spectrum and the upper limit of the UV-C spectrum. The reason is that the inventor has realized that such type of light source can be used in combination with humans and animals without causing harm, in particular if used in combination with one or more sensor for monitoring the light intensity and/or dose from the corresponding light emitting unit(s).

UV light can perform many reactions one of such reactions can be with genetic material through interaction between photons and nucleic acids in a reaction that polymerizes nucleic acids, often forming pyrimidine dimers, such as thymidine dimers. Polymerized bases are harmful to the cell as these cannot be replicated and transcribed. UV light also reacts with proteins by crosslinking amino acids and are thus capable of disabling the function of proteins.

DNA damage is repaired by several mechanisms in organisms. One mechanism is the photoreactivation reaction where the enzyme responsible for the reaction, cleaves the damaged DNA in a reaction with light in the 350-500 nm range. Visible light can in this way salvage damaged bacteria. The photo-reactivation of the bacteria will depend on the light and the exposure time.

UV damage alone can sometimes result in sterile bacteria. For bacteria to be pathogenic, they have to be able to replicate themselves.

Another repair mechanism is the dark repair mechanism. In the dark repair mechanism enzymes can repair damaged DNA without light energy. Such an enzyme is N-glycosylase enzymes that is capable of cleaving N-glycosidic bonds, such that for example deaminated cytosines can be replaced. As organisms can repair DNA damages in multiple ways it is an object of the present disclosure to damage the organisms in such a way, that the bacteria is not reactivated upon reaction with light. This can for example be by a combination of DNA damages and oxidation reactions.

By the present disclosure it is realised that by reducing the microbial pressure in the animal housing, significant reductions in the amount of antibiotics and other medicines can be achieved whereby both the animal health is improved and cost-savings for the farmer are achieved. A further advantage realised by the present disclosure, in particular in relation to farms with pigs and piglets, is that bacteria and viruses (for example MRSA and SADS-COV can be removed significantly and thereby the health and well-being for both animals and farm workers is improved. A reduction in the microbial pressure can be achieved by light emittance and/or liquid disinfection with methods of the present disclosure. Such light emittance and/or disinfection can be inside the animal farm production facilities.

In addition to ensuring healthier animals due to facilitating formation of ND3, thereby strengthening the immune system of the animals, the presently disclosed light emitting unit is preferably further configured to increase the health of the animals by reducing the microbial pressure in the animal farm production facility. The light emitting unit is therefore preferably configured to emit light having a wavelength range that inactivates microbes, such as bacteria and viruses.

This may allow for significant reductions in the amount of antibiotics and other medicines whereby both the animal health is improved and cost-savings for the farmer are achieved. A further advantage realised by the present disclosure, in particular in relation to farms with pigs and piglets, is that MRSA bacteria as well as other aerosol carried infections, such as virus infections, e.g. SADS-COV, can be removed significantly and thereby the health and well-being for both animals and farm workers is improved. A reduction in the microbial pressure can be achieved by disinfection with methods of the present disclosure.

Inactivation of microorganisms is typically carried out by the use of light in the UV-C wavelength range, at 253.7 nm. However, contrary to this, the presently disclosed light emitting unit is in one embodiment configured such that light having a wavelength below 280 or below 270 nm is not emitted, thereby ensuring that humans and animals are not exposed to harmful UV-C rays. The light emitting unit may for example be provided with a sharp edge filter glass where wavelengths below 280 nm or below 270 nm are filtered from the light emitted from the lamp. The UV-C wavelengths are thereby prevented from passing through the sharp edge filter glass. In order to inactivate microorganisms by the use of light having a wavelength of at least 280 nm, the emitted light has a significantly higher energy in the UV-B region, as compared to a standard UV HID lamp. However, in a further embodiment the light emitting unit may be configured to emit monochromatic UV-C light between 215 and 245 nm, more preferably between 218 and 230 nm, most preferably between 217 and 227 nm, for example centred at 222 nm, preferably provided by means of an LED light.

In a preferred embodiment of the present disclosure, the light emitting unit is adapted such that it can be accommodated by a single standard lamp socket or on a single print circuit. The light emitting unit may therefore preferably be configured such that it only has a single connector, that is to be received by said socket.

Therefore, in a specific embodiment of the present disclosure, the light emitting unit is configured to promote the formation of ND3 in the skin of living animals, reduce the microbial pressure in the animal housing, and provide optimized working light conditions, while being adapted such that it can be received by a single standard lamp socket, thereby decreasing the overall costs and space requirements.

In a preferred embodiment of the present disclosure, the light emitting unit is adapted such that it can be accommodated by a single standard lamp socket. The light emitting unit may therefore preferably be configured such that it only has a single connector, that is to be received by said socket.

Therefore, in a specific embodiment of the present disclosure, the light emitting unit is configured to promote the formation of ND3 in the skin of living animals, reduce the bacterial pressure in the animal housing, and provide optimized working light conditions, while being adapted such that it can be received by a single standard lamp socket, thereby decreasing the overall costs and space requirements.

### LED—Light Emitting Diode

The light sources used in the present disclosure is light emitting diodes (LED), hence, the preferred embodiment of the presently disclosed light emitting unit is based on LED technology. An LED and/or LED-chip, such as a surface-mounted-diode or a chip-on-board, is a semiconductor light source that emits light when current flows through the semiconductor. Preferably, single-color LEDs can be configured such that single-color LEDs can emit light in a narrow band of wavelengths from near-infrared through the visible spectrum into the ultraviolet range by selecting different semiconductor materials. The operating voltage of the LED increases by shorter wavelengths due to the larger band gap of these semiconductors. In general, an LED may refer to a light-emitting diode, while a LED-chip may refer to a chip comprising an LED. However, these two terms are used herein interchangeably.

Light from an LED is not coherently monochromatic like the light from a laser, but in the context of this application the separate light emitting diodes are seen as monochromatic light sources, because the emitted wavelength range is indeed monochromatic compared to a broad spectrum polychromatic light source. Hence, the light emitting unit may be configured such that the monochromatic LED light spectrum emitted from the LEDs has a full width half max (FWHM) spectral bandwidth of less than 30 nm, preferably less than 20 nm, more preferably less than 15 nm, yet more preferably less than 10 nm, most preferably around 10 nm, or even around 9, 8, 7, 6 or 5 nm or even smaller.

An UV-B LED centred at 283 nm with a FWHM of 16 nm, will provide approx. 50% intensity around 275 nm, whereas below 268 nm the light emittance is insignificant. Hence, the major advantage of LED is that very specific and narrow wavelength ranges can be targeted, however still with a certain spectral bandwidth such that several wavelengths having certain functional properties can be emitted from the LED.

One advantage of using LED chips is that they are much more efficient in converting electrical power to illumination power, in particular in the UV range. An UV-B LED chip at 283±5 nm rated at 10.5 Watt (i.e. power consumption), can provide more than 3 Watt UV-B light, i.e. a very efficient electrical-to-light energy conversion compared to for example traditional high pressure (HID) UV lamps

Presently the cost of LED UV sources is however higher than high pressure UV lamps, but the running cost of high-pressure lamps will much more quickly increase than LED technology due to the higher electrical power consumption. And in general LED technology is much more environmentally friendly than UV HID technology. In particular LED Chips based on MOCVD (Metal-Organic Chemical Vapor Deposition) has a discharge with relatively high wattage as well as in the desired wavelength ranges of 200 nm to 750 nm. Tests has shown that one of the presently disclosed light emitting units targeted for a pigsty with one sow and 10-15 piglets, and configured for providing 4500 K visible working light for 16 hours per day and UVB and UVC LEDs at 297 nm, 283 nm and 230 nm, respectively, for 24-hour light emittance, can operate with an electrical power consumption of only around 0.3 kWh per day—that is very power efficient.

In one embodiment the presently disclosed light emitting unit is configured for emitting monochromatic UV-B light with wavelength(s) in the range of 275-305 nm, preferably with a maximum intensity at 297±5 nm. The benefits achieved by such UV-B light are that inactivation of bacteria and virus is achieved. In particular it is noted that with LED light with a peak wavelength at around 297 nm, light is provided in a well-defined wavelength spectrum around 297 nm where stimulation of the natural production of ND3 is provided, in particular if the light contains light at one or more wavelengths around 297 nm, 302 and 303 nm. At the same time microorganisms can be inactivated with light at around 295-296 nm, in particular it is an advantage that such light can penetrate small layer of organic material such that inactivation of microorganisms is very efficient. With a peak wavelength at 297±5 nm and FWHM of the wavelength spectrum of around 15 nm, light below 280 nm is substantially avoided. FIG. 9 shows an example of the wavelength spectrum of an LED with a peak wavelength at 295 nm and a FWHM of around 13-14 nm. It is seen from FIG. 9 that if the FWHM is reduced to around 10 nm, the remaining light below 280 nm is insignificant, thereby avoiding those animals and humans in the farm production facility become “sunburnt”, whereas there will still be some light around 290 nm and 300 nm. A further advantage of an UVB LED light source is that no visible light is emitted, such that such a light source can be active for 24 hours without disturbing the sleep of the animals, but at the same time maintaining the advantages in terms of ND3 stimulation and microorganism inactivation.

The presently disclosed light emitting unit may also configured for emitting monochromatic UV-C light with wavelength(s) in the range of 217-227 nm, preferably with a maximum intensity at 222±5 nm. The benefits achieved by such UV-C light are that inactivation of bacteria and virus is achieved very efficiently, because proteins, for example at the surface of a virus, strongly absorb light below 240 nm. The most optimal interval for deterioration of proteins is between 220 and 240 nm. The advantage of 220-240 nm is that such UVC light does not deteriorate cornea and skin of animals and humans in the animal farm production facility. It is quite surprising that UV-C light can be used in this way, because the hitherto known approach has been to reduce or eliminate any UV-C light inside animal farm production facilities. However, with LED technology a suitable wavelength with narrow range light emittance has been identified that can efficiently reduce the microbial pressure inside the animal farm production facility without harming animals or humans. In particular it has been shown to be advantageous to include UVC light at 222 nm and/or 233 nm. Light at 222/233 nm has been shown to be very efficient for deteriorating bacteria because DNA can be severely damaged at 222/233 nm. A further advantage of an UVC (220-240 nm) LED light source is that no visible light is emitted, such that such a light source can be active for 24 hours without disturbing the sleep of the animals

With LED technology the colour temperature and intensity of the visible light can be accurately customized to improve the working light conditions of the humans and disturb the animals as little as possible. In that regard it has turned out that redshifting the light is advantage in animal farm production facilities, i.e., the presently disclosed light emitting unit may be configured such that the colour temperature of the visible light is in the range 4500-6500 K or in the range of 4000-6000 K, most preferably primarily at 4500 K corresponding to a wavelength spectrum centred at 644 nm. Such visible light LEDs can be provided in wattage from around 1 W to more than 1000 W. Hence, visible light emittance can easily be selected in accordance with the application and situation, in particular the size of the animal farm production facility and where the light emitting units are located.

The visible light may be provided by a single diode, however more diodes can be provided if necessary. The monochromatic UV-B light can be provided by at least one, two, three or four LEDs. Similarly, the monochromatic UV-C light can be provided by at least one, two, three or four LEDs.

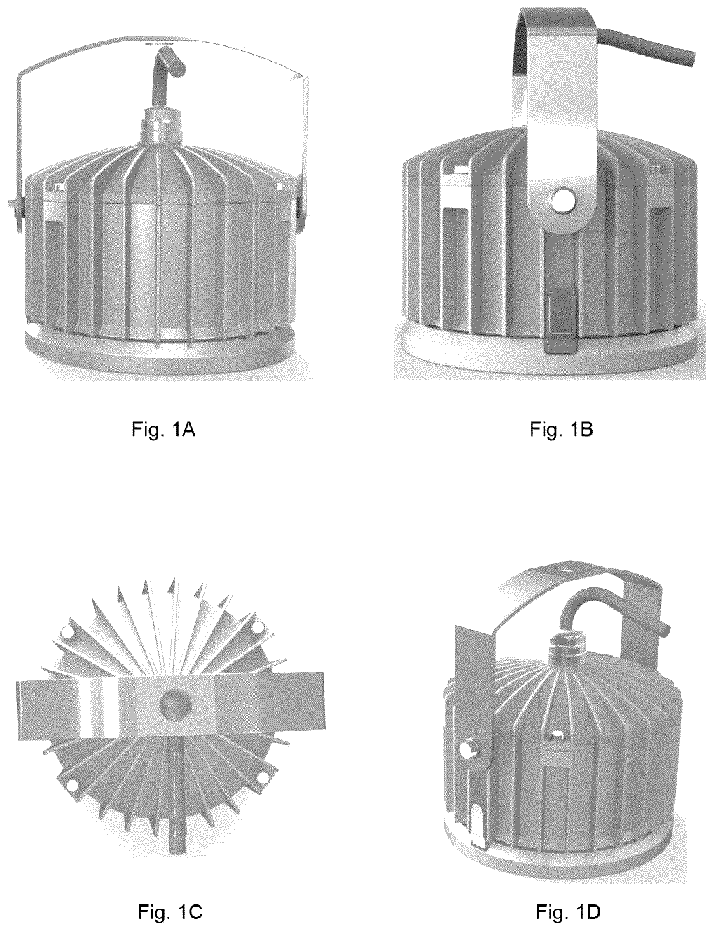

All LEDs can be selected and combined in different wattage, selected in accordance with the required light intensity. LEDs are available from around 1 watt to around 50 watts, for example 1 W, 3 W 12 W and 48 W LEDs. As also stated above LEDs for providing white light/visible light are available in even higher power settings. In accordance with the application and the environmental conditions in the farm production facility the presently disclosed light emitting units can be provided with passive and/or active cooling. Active cooling for example in the form of Peltier elements(s) mounted adjacent the LED circuit board, passive cooling in the form of cooling fins on the housing, as exemplified in FIGS. 1-3.

All LEDs can be mounted on a common circuit board, in a DIL (dual in-line) setup by means of DIL fitting, for example according to CIE 62471. Preferably the light emitting unit is built such that the common circuit board with the LEDs is replaceable. Hence, when the light source need replacement, the user or service technician can mere replace the circuit board.

The light emitting unit may comprise a reflector to spread the light emitted from the LEDs, the reflector can for example be mounted above the common circuit board. The light emitting unit may further comprise a housing, preferably metallic, having cooling fins, such that the light sources are efficiently cooled during operation.

One group of animals very sensitive to the lack of vitamin ND3 is new-born piglets which are born with no measurable level of ND3 in their blood, and hence have a weakened immune system. New-born piglets are dependent on receiving ND3 through the breastmilk from the sow. However, the content of ND3 in the breastmilk is very low. In nature this is not a problem as the feral pigs give birth to their piglets in the summer in which the need for ND3 is entirely covered by the UV-B radiation from the sun. The lack of ND3 plays a key role in the domesticated piglets' ability to fight infections and hence to the mortality of piglets in conventional production. The use of UV light to enhance the formation of ND3 is described in EP 2558984.

### ND3 Formation in Animals

The immune system is the body's defence against foreign organisms, primarily bacteria, fungi, viruses and parasites. The body's immune system comprises millions of different white blood cells, each of which can recognize one particular form of foreign cells. When a foreign cell, for example a virus, enters the body, the white blood cells will attack and try to kill the foreign cells. It is therefore crucial in transplants that the tissues that are inserted resemble as far as possible the patient's own cells. The immune system has a memory, so the next time it is exposed to the same type of bacteria or virus, it will have built antibodies against this particular bacterium or virus and can quickly eradicate it. The immune system is divided into an innate and an adaptive immune system.

The adaptive immune response is permanent and antigen-dependent. The presently disclosed combination of UV LED light greatly enhances the adaptive immune response.

The level of vitamin ND3 is generally too low in humans and leads to higher mortality rates. It is therefore important to have UV lighting that increases ND3 in the plasma and a high natural vitamin ND3 in food, especially dairy products. Vitamin ND3 is for example an incredibly important factor for new-born babies, as well as for the development of children and young people. Vitamin ND3 deficiency may lead to respiratory infections, asthma, osteoporosis and other secondary diseases. Studies also indicated more than 30% of Type 1 diabetes cases and asthma can be prevented if infants receive the recommended dose of vitamin ND3. Indications also suggest that ND3 strengthens the immune system to reduce the risk of breast, prostate and colon cancer and in general reduce the risk of lifestyle diseases, such as cardiovascular diseases and osteoporosis.

A further purpose of the present invention is therefore to provide a light emitting unit that is suitable for enhancing the ND3 formation in the skin of an animal, such as a new-born piglet. The presently disclosed light emitting unit serves as a way to ensure the vitamin ND3 formation in the skin of the animal.

The most effective irradiation of 7-dehydrocholesterol to pre-vitamin D3 generation comes from 297 nm UV-B light. The most effective wavelength for the conversion of the keratinocytes to pre-vitamin D3 is 302 nm UV-B light. Furthermore, a secondary maximum below 285 nm is seen in some situations.

Preferably the light emitting unit is configured to emit light comprising wavelengths in the range 280-305 nm, which is part of the UV-B range. This range turns out to be most efficient for stimulating the natural formation of ND3 in piglets. It is a further preference that the light emitting unit comprises a high intensity for light having a wavelength of 295 nm, more preferably 297 nm. It has been shown that light of high intensity in the wavelength of 297 nm is particularly well suited for the formation of ND3 in the skin of an animal.

Illumination on an animal such as a piglet will result in a more effective formation of ND3 in the skin of the animal. This will result in healthier animals and decreasing mortality within a population of animals. Another desirable effect of these health improvements is that it enables the breeder to reduce the use of antibiotics which will be an important step to avoid development of multi-resistant bacteria. Another positive effect of an enhanced formation of ND3 is a better absorption of the phosphorus and calcium in the animal feed which will have a positive environmental effect for the production of feed. Farm animals with a high content of ND3 in their body produce milk and meat with a higher level of ND3 content and serve as an improvement of the human's diet-based vitamin D3 absorption.

### Lighting System

The present disclosure further relates to a system, e.g., a lighting system, such as a modular lighting system, for 1) reducing the microbial pressure, and/or 2) stimulating the production of natural vitamin D3, and/or 3) providing working light, in a room for accommodating humans and/or animals, such as a classroom, a hospital ward, an office space, an assembly hall, an animal farm production facility, etc the system comprising:
at least one of the presently disclosed light emitting units, and
a control system adapted for managing and/or controlling
a. light exposure time, and/or
b. light exposure intensity, such as total light emittance
i. in selected wavelength ranges of the light emitting unit(s), i.e. the selected wavelength ranges disclosed herein.

As the presently disclosed light emitting unit may be suitable for emitting light in a plurality of wavelength ranges, e.g., by means of a plurality of different LEDs, the control system may be adapted for controlling each LED separately, e.g., in terms of power adjustment to adjust the intensity of emitted light and in terms of light exposure time per minute, hour, day, week, month, year, etc.

The lighting system may comprise at least one light sensor configured for measuring the light exposure from the light emitting unit(s), either sensors that can be measure both visible light and UV-B and/or UV-C light, or separate sensor for separate wavelength ranges. Sensor(s) may be placed near corresponding light emitting unit(s), e.g. incorporated in a housing thereof; additionally or alternatively configured for being placed in the same room, e.g. for mounting on a wall, such that the sensor(s) can be illuminated by light from the light emitting unit(s). The system may be configured to turn off at least the UV-B LEDs and/or UV-C LEDs when light exposure sensors are covered or somehow malfunction.

LEDs typically degrade over time such that the emitted intensity falls slowly over time for the same power, typically 30% over 5000 hours, or even 10.000 hours. By measuring the light intensity form the light emitting unit(s) it can be ensured that it is the emitted light intensity that is the control parameter. Hence, the lighting system may be configured for maintaining emittance of a predefined light exposure intensity in selected wavelength ranges of the light emitting unit(s) based on measurements of the light exposure from the light emitting unit(s). In that regard the light emitting unit can be configured such that the LEDs are adjusted to full power when new, but instead ramped up slowly in correspondence with the slow degrade in efficiency. A much longer lifetime of the individual LEDs can then be achieved. The LED used can have be specified to have a typical lifetime of 5000 hours with a decay in emittance of 30% over the lifetime. By ramping the power over a long period of time, this decay can be compensated and the lifetime of the LED chips can be increased.

A control parameter can also be the relative intensity of the various UV-B and/or UV-C LED wavelengths relative to each other, as also discussed in here.

The lighting system may be advantageously be configured such that the light emitting units only emit UV-C light when humans and/or animals are not present in vicinity of the light emitting unit(s), such as in a corresponding room containing the light emitting system. E.g. configured such that the light emitting units only emit UV-C light when humans are not present in the room, as UV-C light can be harmful to humans and animals. This can be provided by different means.

The lighting system may be configured such that the light emitting unit(s) only emit UV-C light during a selected period during the day, e.g. during closing time of the room, such as night-time, for example from 22 to 5 o'clock. Closing time of the specific room or building can be defined by a user/administrator such that the control system can control the light emitting unis(s) accordingly.

The lighting system may comprise and/or be in contact with at least one movement sensor for detecting activity in the room and/or the building. This is another way to ensure that harmful light is not emitted from the light emitting unit(s) when humans are present.

Sensors which is part of or merely in contact with the system, can be connected to the system by wireless connection, or by wire if required.

In one embodiment the presently disclosed light emitting unit and/or system is configured such that visible light is emitted for a limited and predefined period per day, e.g. between 8 and 16 hours per day, whereas non-visible light, in particular UVB and/or UVC is emitted for 24 hours per day, because these non-visible light sources have functional properties in terms of inactivation of microorganisms and/or stimulation of NDS in animals. Thereby maximization of the functional light sources can be obtained without disturbing the animals' sleep, e.g. during night.

In one embodiment the presently disclosed light emitting unit and/or system is configured such that to ramp up the light intensity of one, more or all of the UV LEDs. This can in particular be provided to avoid “sunburn” of human or animals in the corresponding room. The ramping of the light intensity is typically defined by an initial power setting, a stepwise increase, a duration at each power setting and a maximum power setting. A ramp is typically defined over a few hours or even over a whole day or several days.

The presently disclosed light emitting unit and/or system may further be configured such that it can be controlled, e.g. remotely, such as from a smartphone or another display device, which light sources/wavelength spectra that are active and possibly also the corresponding intensity/power. Possibly the timing of the different light LEDs can be controlled remotely. Individual control of light sources/wavelength spectra is particular an option with LEDs. Hence, the presently disclosed light emitting unit and/or system may be configured for emittance of broadband visible light for a first predefined period of time per day, such as less than 20, 18, 16, 12 or 8 hours per day, and configured for emittance of monochromatic non-visible LED light for a second predefined period of time per day, such as at least 20 hours, or 22 hours or even 24 hours. Hence, preferably the second predefined period of time is larger than the first predefined period of time.

The presently disclosed light emitting unit and/or system may further be configured to control a ratio of light emittance, such as based on input from sensor(s), between the at least second UV-B LEDs and the at least first UV-B LED(s) such that the ratio of total light emittance of 283±5 nm light relative to total light emittance of 297±5 nm light can be selected.

### EXAMPLES

In the following the present disclosure is described with reference to preferred embodiments and the accompanying drawings.

### Example 1-Disinfection Efficiency of UV LED Irradiation on Staphylococcus aureus Inoculated on Glass Beads

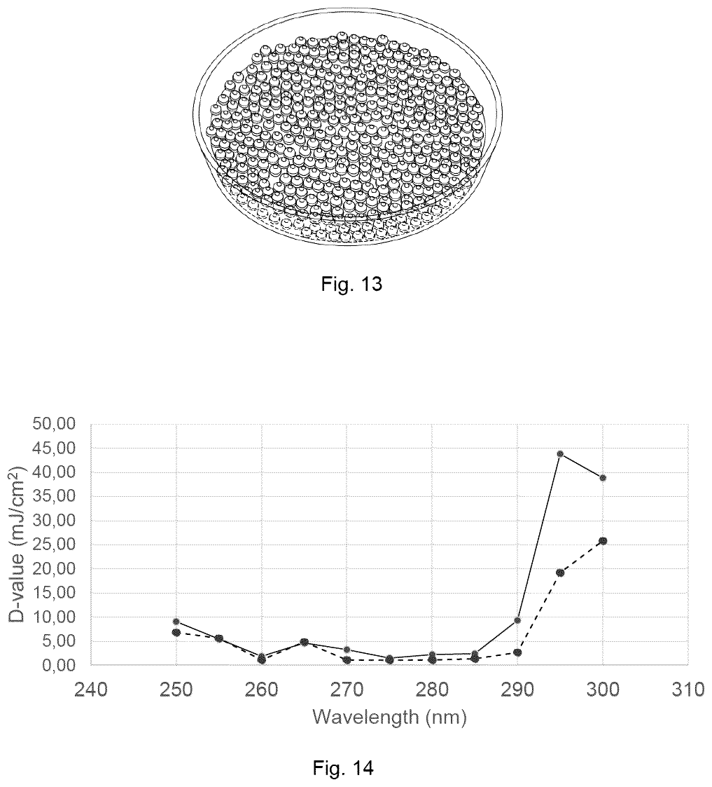

The aim of this experiment was to test the disinfection efficiency of UV LED irradiation on Staphylococcus aureus (S. aureus) at different contact time. In a sterile petri-plate, S. aureus was inoculated on sterile glass beads and exposed to UV irradiated at different contact time. FIG. 13 is an illustration of the glass beads in a petri dish used in this example, the glass beads are approx. 6-8 mm in diameter.

Different embodiments of the presently disclosed UV LED light emitting unit was used in different experiments. In each experiment one UV LED light emitting unit was mounted horizontally 76 cm above the petri-plate containing the S. aureus inoculated glass beads. At different contact time of UV irradiation, S. aureus inoculated petri plate was removed and placed in the dark. Similarly, two petri-plate containing S. aureus inoculated glass beads were kept in the dark (away from UV irradiation) which were later used to enumerate the bacteria at time zero.

After the end of the UV irradiation (24 h), S. aureus from each petri-plate were enumerated using a plate count method in mannitol salt agar (MSA). After 24 h incubation at 37° C., the number of colonies observed in the MSA plate was used to calculate the removal of S. aureus from UV irradiation at different times.

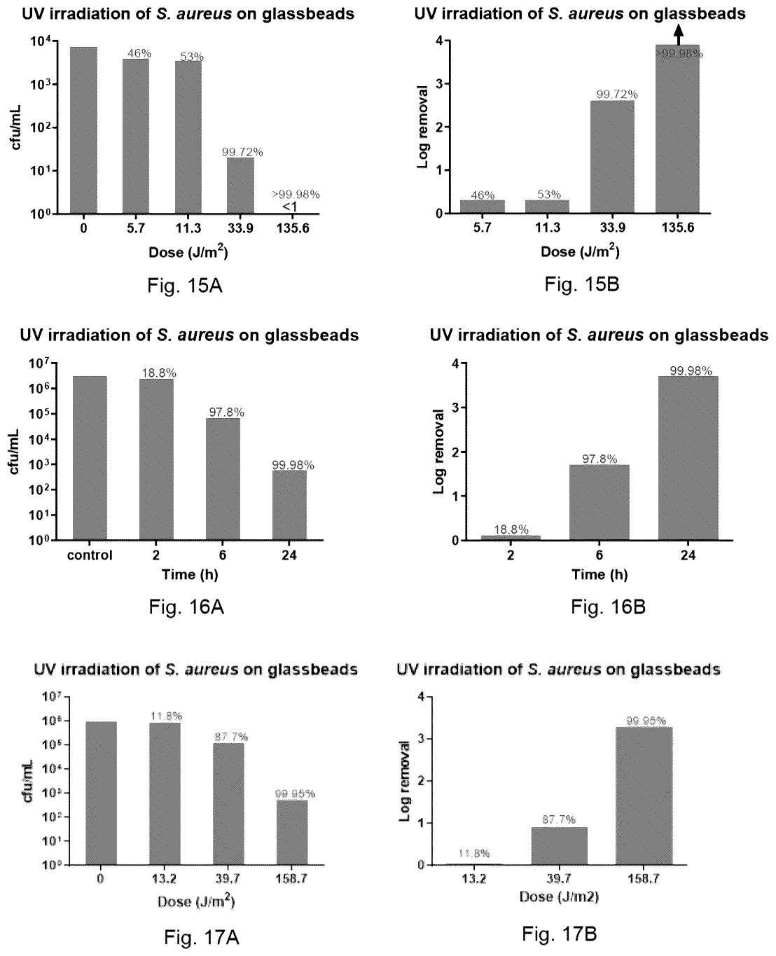

Tables 1-3 below shows the percentage removal of S. aureus bacteria on glass beads at different times during UV LED irradiation. This is also illustrated in FIGS. 15-17.

TABLE 1
285 + 295 nm with FWHM of 15 nm

| Time (hours) | cfu/mL | Log removal |
|---|---|---|
| 0 | 7150 | x |
| 1 | 3893 | 0.3 |
| 2 | 3560 | 0.3 |
| 6 | 20 | 2.6 |
| 24 | <1 | >3.9 |

TABLE 2
280 + 297 nm with FWHM of 15 nm

| Time (hours) | cfu/mL | Log removal |
|---|---|---|
| 0 | 2960000 | x |
| 2 | 2405000 | 0.1 |
| 6 | 66000 | 1.7 |
| 24 | 555 | 3.7 |

TABLE 3
285 nm with FWHM of 40 nm

| Time (hours) | cfu/mL | Log removal |
|---|---|---|
| 0 | 1015000 | x |
| 2 | 895000 | 0.05 |
| 6 | 125000 | 0.91 |
| 24 | 525 | 3.3 |
| 48 | <1 | 6.0 |

The “285±295 nm” used different UV-B LEDs at 285 nm (2.5 mW) and 295 nm (4 mW), both with FWHM of approx. 15 nm, weighted dose per 8 hours of 32.5 J/m², see also FIG. 15A showing the amount of bacteria with bars on log scale vs. the dose (J/m²), where 5.7 corresponds to one hour, 11.3 corresponds to 2 hours, and so on like the times in table 1. The percentage removal of bacteria is shown in numbers above the bars. FIG. 15B corresponds to FIG. 15A but illustrates the log removal.

The “280±297 nm” used different UV-B LEDs at 280 nm (2.5 mW) and 297 nm (4 mW), both with FWHM of approx. 15 nm, weighted dose per 8 hours of 32.1 J/m², see also FIG. 16A showing the amount of bacteria with bars on log scale vs. the time, like the times in table 2. The percentage removal of bacteria is shown in numbers above the bars. FIG. 16B corresponds to FIG. 16A but illustrates the log removal.

The “285 nm” used a single 285 nm (2.5 mW) UV-B LED, however with a FHWM og approx. 40 nm, weighted dose per 8 hours of 31.9 J/m², see also FIG. 17A showing the amount of bacteria with bars on log scale vs. the dose (J/m²), where 13.2 corresponds to two hours, 39.7 corresponds to 2 hours, and 158.7 corresponds to 24 hours. The percentage removal of bacteria is shown in numbers above the bars. FIG. 17B corresponds to FIG. 157 but illustrates the log removal.

Weighted values according to IEC and SED standards have also been calculated for these experiments and are shown in table 4 below.

TABLE 4
Weighted values of removal of S. aureus inoculated
on glass beads at different dose of UV irradiation

| Dose | 285 + 295 nm | 280 + 297 nm | 285 nm |
|---|---|---|---|
| 1 hour/3.8 J/m² | 31.7% | 30.7% | 18.2% |
| 8 hours/30 J/m² | 95.3% | 93.6% | 77.8% |
| 24 hours/90 J/m² | 99.99% | 99.97% | 98.91% |

As seen from tables 1-4 and FIGS. 15-17 the combination solution of using two UV-B LEDs is very efficient for killing bacteria. The single wavelength more broadspectred 285 nm UV-B LED is a more cost-efficient solution, also suitable for disinfection, however, less efficient compared to the combination solution.

### Example 2

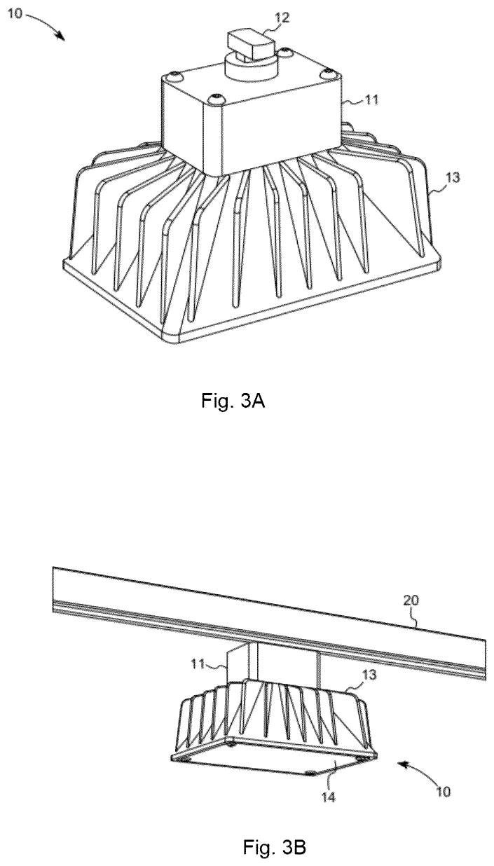

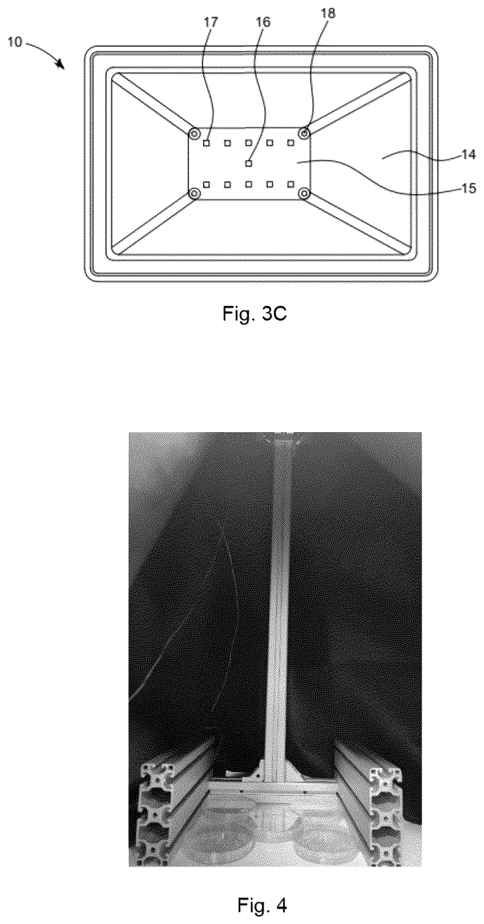

FIGS. 3A-C show one embodiment of the presently disclosed light emitting unit based on LED technology. The light emitting unit 10 comprises a metallic rectangular housing accommodating a power supply 11 inside a top part of the housing, a connector 12 that can engage with a power strip located near the ceiling of a farm production facility, a transparent cover 14 and a plurality of cooling fins 13 to increase the surface of the housing such that it can better cool the LED light sources 16, 17. FIG. 3A shows a perspective view of the light emitting unit 10. FIG. 3B shows a perspective bottom view of the light emitting unit 10 electrically engaged with and hanging from a power strip 20, such that light emittance is down toward animals on the floor of the farm production facility. FIG. 3C shows a bottom view of the light emittance unit 10 where the printed circuit board 15 is seen accommodating eleven LED chips, with a central LED 15 providing neutral white light and the ten remaining monochromatic LED chips 17 in various wavelengths. As seen in FIG. 3C the common printed circuit board is directly accessible from the bottom of the light emitting unit 10 and can thereby be easily replaced by means of four screws 18.

### Example 3

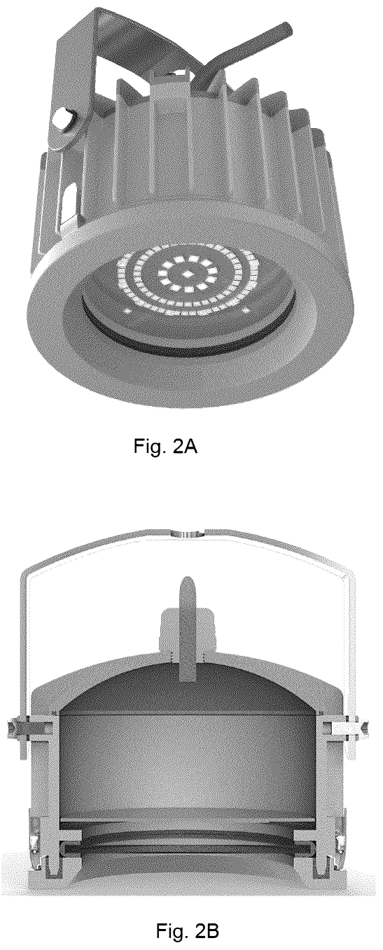

FIGS. 1A-D and 2A-B show one embodiment of the presently disclosed light emitting unit based on LED technology with FIGS. 1A and 1B showing side views, FIG. 1C showing a top view and FIG. 1D showing a perspective top/side view. FIG. 2A shows a perspective bottom view where at least part of a common circuit board 15 is seen accommodating LED chips. FIG. 2B shows a cut-through perspective view of the light emitting unit. The light emitting unit in FIGS. 1 and 2 comprises a metallic circular housing accommodating a power supply inside a top part of the housing and a mounting element for attachment to a ceiling element. The mounting element are attached to the circular housing in a way that enables tilting of the light emitting unit after attachment to a ceiling element. As in FIG. 2 a plurality of cooling fins are provided to increase the surface of the housing such that it can better cool the LED light sources. As seen in FIG. 3C the common printed circuit board is directly accessible from the bottom of the light emitting unit 10 and can thereby be easily replaced by means of four screws 18. The circular housing can advantageous because light from an LED chip is typically emitted in a circular cone angle of approx. ±60 degrees.

### Example 4

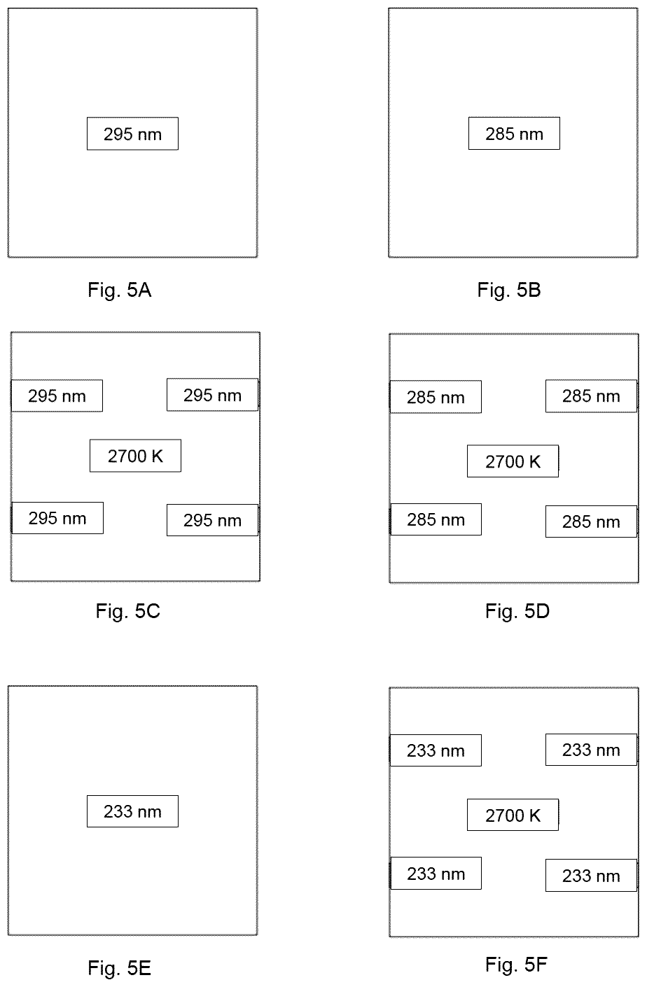

FIGS. 5A-D show various combinations of UV-B and white light LED chips on a common printed circuit board for use in the presently disclosed light emitting unit, in particular suitable for use in animal farm production facilities such as pig houses.

FIG. 5A shows a single UV-B LED 295 nm chip arranged centrally on the circuit board, whereas FIG. 5B shows a single UV-B LED 285 nm chip is arranged centrally on the circuit board. Such a setup is primarily targeting a black light emitting unit for animal nests, e.g. piglet nests and mink nests, where the UV-B light enhances the natural formation of ND3, but where no visible light must be emitted. In an animal nest the light emitting unit is typically located quite close to the animals and a single LED chip might be sufficient.

FIGS. 5C and 5B show combinations of a centrally located polychromatic white light LED chip with a colour of 2700 K and four UV-B LED chips providing monochromatic light at 295 nm in FIG. 5C and 285 nm light in FIG. 5D. A single white light LED chip is sufficient to provide the visible working light for humans in the farm production facility, but if the light emitting unit is located near the ceiling in the facility it will be suitable with four UV-B led chips to provide sufficient light intensity.

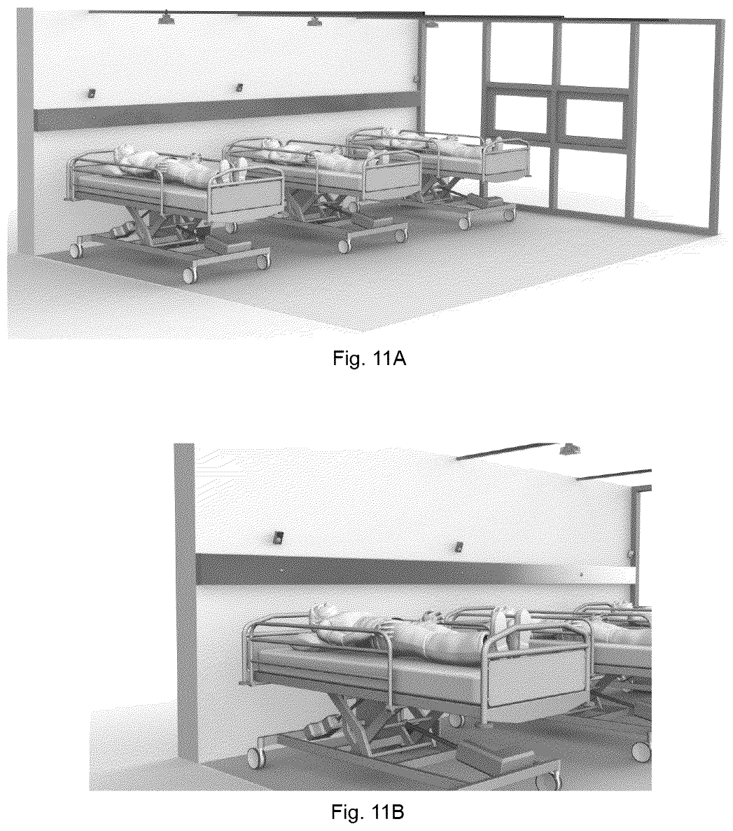

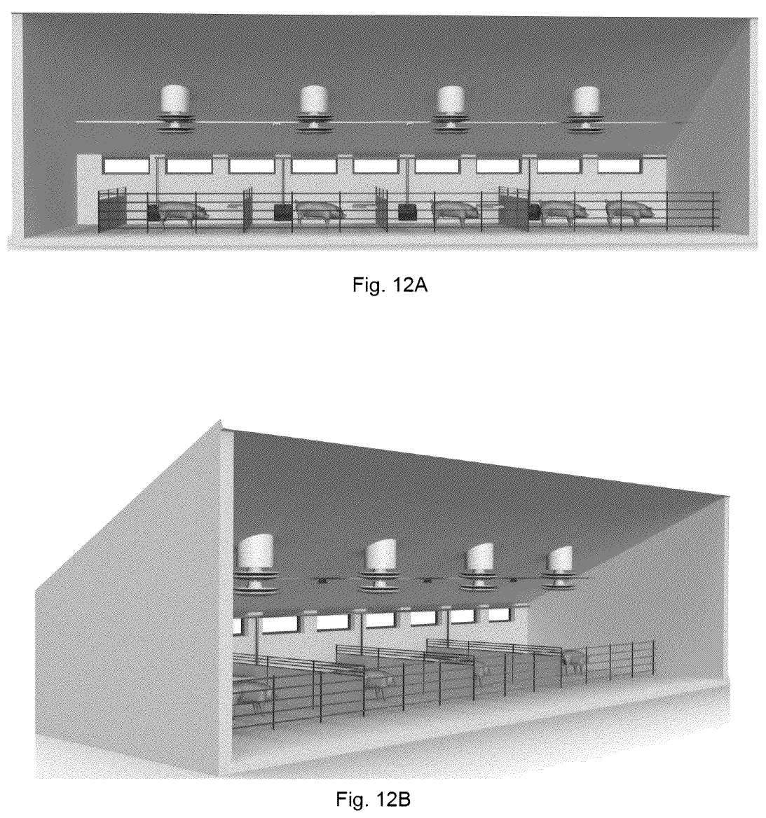

The single UV-B wavelength setups in FIGS. 5A-D can advantageously be provided as broad spectral LED chips, i.e. with a FWHM of around 30 nm, in order to cover and take advantages of the entire UV-B spectre for illumination of the pigs and for reducing the microbial pressure in the pig house, as exemplified in FIG. 12.

### Example 5

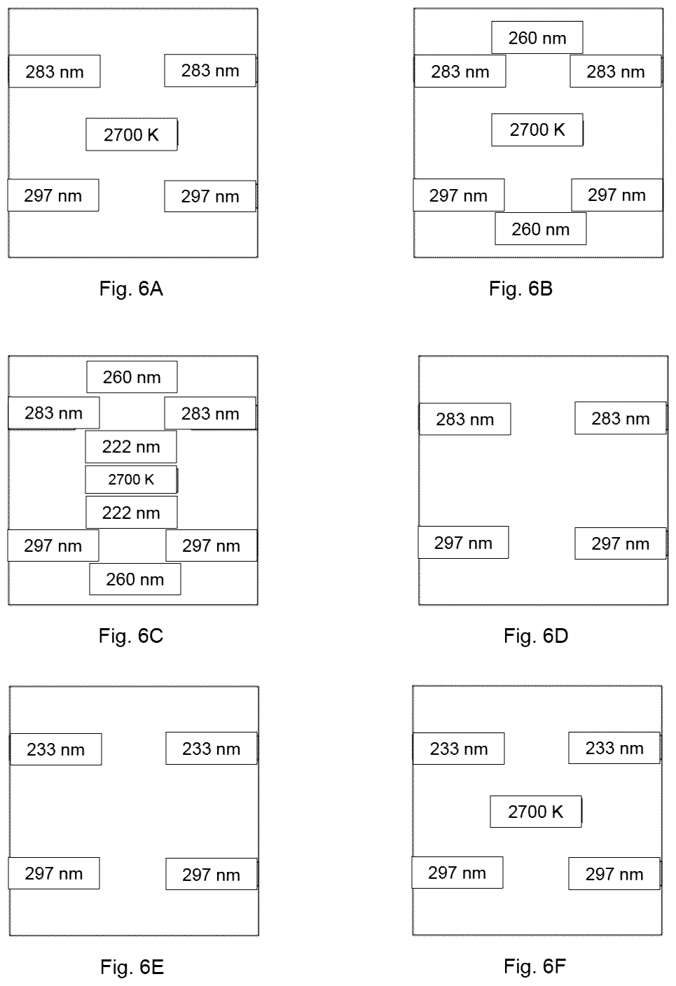

FIGS. 6A-F show various combinations of UV-B, UV-C and white light LED chips on a common printed circuit board for use in the presently disclosed light emitting unit, in particular suitable for use in human facilities, such as hospitals, schools, etc.

The setup in FIG. 6A provides a single white light LED chips with a colour temperature of 2700 K and two 283 nm LED chips and two 297 nm LED chips. Such UV-B chips are typically specified as 283±5 nm and 297±5 nm, respectively, i.e. there can be some variation of the centre wavelength. The typical FWHM is around 10-15 nm. The setup in FIG. 6A is particular suitable for use in human facilities, such as hospitals, schools, etc., because a large part of the UV-B spectrum is utilized, e.g. for generation of ND3 and for reducing the microbial pressure. The advantage in combining 283 and 297 nm is that the ratio of 283 to 297 nm light can be adjusted, either by the relative number of the different LED chips used on the board or by controlling the power supplied to the chips and possibly also in combination with light sensors, e.g. as part of the light emitting unit and/or a part of a light system and e.g. installed on the wall of hospital ward, as exemplified in FIG. 11.

FIG. 6D corresponds to the setup in FIG. 6A, however without a white light source, i.e. two 283 nm LED chips and two 297 nm LED chips. The advantages of the setup in FIG. 6D are the same as for the setup in FIG. 6A, however without the possibility of using the setup in FIG. 6D for providing working light. The setup in FIG. 6D can be used in for example hospitals, schools, and animal farm production facilities as a light emitting unit that is constantly turned on in order to continuously reduce the microbial pressure and stimulate production of ND3, however without being visible to the human or animal eye, i.e. during the night the animals can sleep undisturbed.

FIG. 6B corresponds to the setup in FIG. 6A and further supplied with two 260 nm UV-C LED chips that can further reduce the microbial pressure. However, the light from such UV-C LED chips at 260 nm are harmful for humans and animals and should only be used when no humans or animals are present.

FIG. 6C corresponds to the setup in FIG. 6B and further supplied with two 222 nm UV-C LED chips that can further reduce the microbial pressure. The light from such UV-C LED chips at 222 nm can actually be used in combination with humans, but should be used with case and preferably in combination with one or more sensors that can monitor the dose provided by such UV-C light.

The setup in FIG. 6E two 233 nm UV-C LED chips and two 297 nm UV-B LED chips. Such LED chips are typically specified as 233±5 nm and 297±5 nm, respectively, i.e. there can be some variation of the centre wavelength. The typical FWHM is around 10-15 nm. The setup in FIG. 6E is particular suitable for use in human facilities, such as hospitals, schools, etc., because the 297±5 nm UV-B light is suitable for generation of ND3 and also partly for reducing the microbial pressure, whereas the 233±5 nm UV-C is very efficient for reducing the microbial pressure without causing harm to humans or animals. The advantage in combining 233 and 297 nm is that the ratio of 233 to 297 nm light can be adjusted, either by the relative number of the different LED chips used on the board or by controlling the power supplied to the chips and possibly also in combination with light sensors, e.g. as part of the light emitting unit and/or a part of a light system and e.g. installed on the wall of hospital ward, as exemplified in FIG. 11. The setup in FIG. 6E can be used in for example hospitals and schools, as a light emitting unit that is constantly turned on in order to continuously reduce the microbial pressure and stimulate production of ND3, however without being visible to the human eye, i.e. during the night the patients can sleep undisturbed.

The setup in FIG. 6F corresponds to the setup in FIG. 6E with the further addition of a white light LED with a colour temperature of 2700 K, such that working light also can be provided.

If limiting zoonoses in the whole farm production facility is the primary purpose, e.g. for swine flu which may turn into a pandemic, the presently light emitting unit can be configured with at least one UVB LED chip for at least one UVC LED Chips for 24 hour illumination and at least one visible light LED for providing work light at around 2700 Kelvin, e.g. for 16 h illumination. The number of LEDs and their wattage is selected in accordance with the specific condition in the farm production facility.

If the location of the light emitting unit is more local, i.e., in a pigsty, the wavelength and power configuration must be selected depending on the size, age and/or type/race of the animals, for example whether it is piglets, weaners or slaughter pigs. In the pigsties that need for visible light may be reduced to less hours per day, whereas the UVB and UVC light is still important for 24 hour illuminations to maximize the positive effect. But the number and/or wattage of the LED chips may be reduced, in particular in the animals are young and/or the light emitting unit is located closer to the animals.

### Example 6

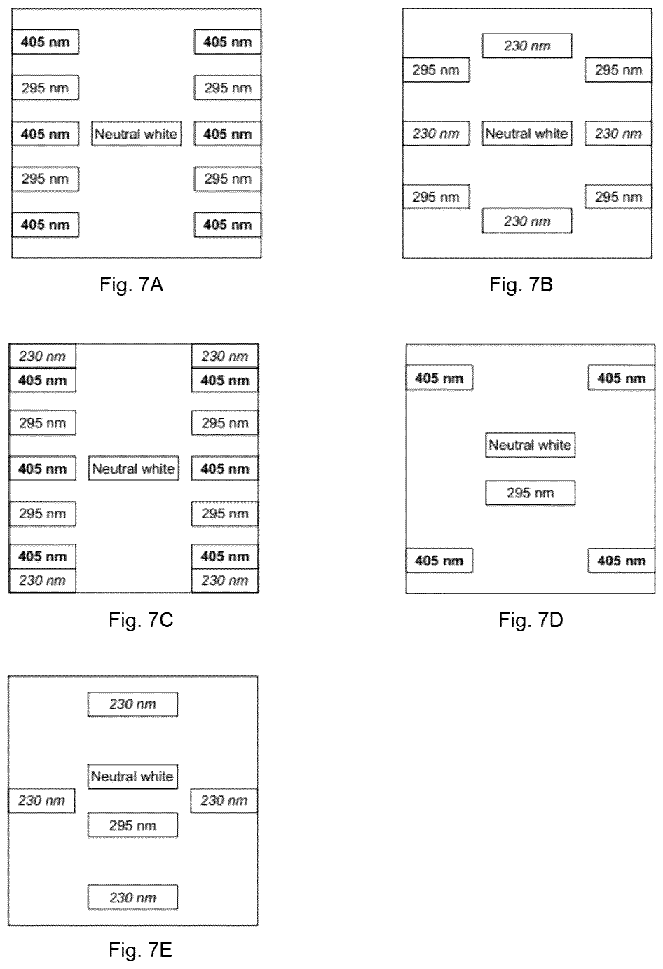

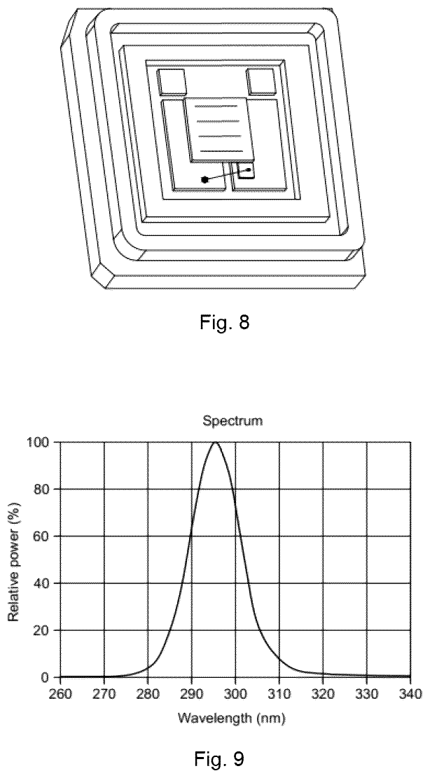

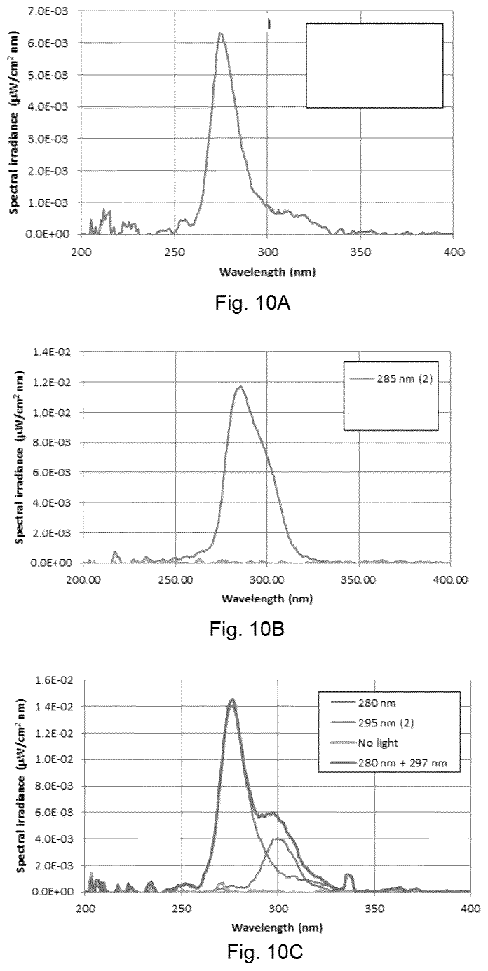

FIGS. 7A-E show various combinations of UV-B, UV-C, and white light LED chips on a common printed circuit board. The LED chips can for example be 3-watt chips 80-watt chips, or anything in between.

In FIG. 7A the circuit board in FIG. 5C is supplied with six LED chips providing monochromatic light at 405 nm to further reduce the microbial pressure in the farm production facility.

In FIG. 7B the circuit board in FIG. 5C is supplied with four LED chips providing monochromatic UV-C light at 230 nm to further reduce the microbial pressure in the farm production facility.

In FIG. 7C the circuit board in FIG. 7A is supplied with four LED chips providing monochromatic UV-C light at 230 nm to further reduce the microbial pressure in the farm production facility. The setup in FIG. 7C accommodates as many as fifteen LED chips: One chip for the visible working light, four UV-C chips, four UV-B chips and six LED chips providing 405 nm light, corresponding to dark violet.

FIG. 7D provides a single white light LED, a single 295 nm LED chips and four 405 nm LED chips. In FIG. 7E the 405 nm LED chips are replaced with 230 nm UV-C LED chips (relative to FIG. 7D) in order to increase sterilization capability.

The examples in FIG. 7 with only one UV-B LED chip in all setups illustrate that less UV-B intensity might be necessary, for example if the primary purpose of the UV-B light is to stimulate production of natural ND3 in the animals, whereas high intensity is needed from the UV-C and violet light sources to maximize reduction of the microbial pressure inside the farm production facility.

### Example 7

FIG. 5E shows a single UV-C LED 233 nm chip arranged centrally on the circuit board. Such a setup is primarily targeting disinfection applications in substantially clean environments, e.g. hospitals, schools, etc. Light around 230 nm can be used on humans with very little risk of harm, i.e. the microbial pressure can be reduced 24/7 without emitting visible light. Light around 230 nm can also in principle be used in animal farm production facilities, because the disinfection capability is also applicable there and the light causes no harm to the animals. However, dust and fine particles present in animal farm production facilities may absorb the 233 nm light, in particular if present on the skin of the animals, and thereby significantly reduce the disinfection capability of the UV-C light.

FIG. 5F shows a combination of a centrally located polychromatic white light LED chip with a colour of 2700 K and four UV-C LED chips providing monochromatic light at 233 nm. A single white light LED chip is sufficient to provide the visible working light for humans, and if the light emitting unit is located near the ceiling in the premises it will be suitable with four UV-C led chips to provide sufficient light intensity for disinfection.

### Items

A light emitting unit for 1) reducing the microbial pressure, and 2) stimulating the production of natural vitamin D3, the light emitting unit comprising
a. at least one UV-B Light Emitting Diode (LED) configured for emitting monochromatic UV-B light.

The light emitting unit according to item 1, comprising at least a second UV-B LED configured for emitting monochromatic UV-B light having a maximum intensity between 280-290 nm, more preferably between 283-287 nm, most preferably at 285 nm.

The light emitting unit according to item [00151], wherein the monochromatic UV-B LED light has a full width half max (FWHM) spectral bandwidth of less than or equal to 50 nm, more preferably less than or equal to 40 nm, even more preferably less than or equal to 30 nm, most preferably less than or equal to 20 nm.

The light emitting unit according to item [00151], wherein the monochromatic UV-B LED light has a full width half max (FWHM) spectral bandwidth of at least 30 nm, more preferably at least 35 nm, most preferably at least 40 nm.

The light emitting unit according to item 1, comprising
a. at least a second UV-B LED configured for emitting monochromatic UV-B light having a maximum intensity between 278-288 nm, more preferably between 281-285 nm, most preferably at 283 nm.

The light emitting unit according to any of the preceding items, comprising
a. at least a first UV-B LED configured for emitting monochromatic UV-B light having a maximum intensity between 292-302 nm, more preferably between 295-299 nm, most preferably at 297 nm.

The light emitting unit according to any of items [00154]-[00155], wherein the monochromatic UV-B LED light has a full width half max (FWHM) spectral bandwidth of less than or equal to 30 nm, more preferably less than or equal to 20 nm, even more preferably less than or equal to 15 nm, most preferably less than or equal to 10 nm or even 8 nm.

The light emitting unit according to any of the preceding items [00154]-[00156], configured to control a ratio of light emittance between the at least second UV-B LED and the at least first UV-B LED.

The light emitting unit according to any of the preceding items [00154]-[00157], wherein a ratio of light emittance between the at least second UV-B LED and the at least first UV-B LED is more than 1, preferably more than 2, more preferably more than 2.5, most preferably around or more than 3.

The light emitting unit according to any of the preceding items [00154]-[00157], wherein a ratio of light emittance between the at least second UV-B LED and the at least first UV-B LED is less than 1, preferably less than 0.75, most preferably less than 0.5.

The light emitting unit according to any of the preceding items, comprising
a. at least one UV-C LED configured for emitting monochromatic UV-C light.

The light emitting unit according to item [00160], wherein the at least one UV-C LED is configured for emitting monochromatic UV-C light with wavelengths in a range of 215-240 nm.

The light emitting unit according to any of preceding items [00160]-[00161], comprising
a. at least a first UV-C LED configured for emitting monochromatic UV-C light having a maximum intensity between 228-238 nm, preferably between 231-235 nm, most preferably at 233 nm.

The light emitting unit according to any of preceding items [00160]-[00162],
a. at least a second UV-C LED configured for emitting monochromatic UV-C light having a maximum intensity between 217-227 nm, preferably between 220-224 nm, most preferably at 222 nm.

The light emitting unit according to any of preceding items [00160]-[00163],
a. at least a third UV-C LED configured for emitting monochromatic UV-C light having a maximum intensity between 255-265 nm, preferably between 258-262 nm, most preferably at 260 nm.

The light emitting unit according to any of preceding items [00160]-[00164], wherein the monochromatic UV-C LED light has a full width half max (FWHM) spectral bandwidth of less than or equal to 20 nm, more preferably less than or equal to 15 nm, even more preferably less than or equal to 10 nm, most preferably less than or equal to 8 or even 5 nm.

The light emitting unit according to any of the preceding items [00160]-[00165], comprising at least one UV-B LED and at least one UV-C LED and configured to control a ratio of light emittance between the at least one UV-B LED and the at least one UV-C LED.

The light emitting unit according to any of the preceding items, comprising at least one visible light LED configured for emitting polychromatic visible light, preferably with wavelengths in the range of 380 nm-750 nm.

The light emitting unit according to any of the preceding items, configured for not emitting light below 270 nm, except emittance of monochromatic UV-C light in a range of 215-240 nm.

The light emitting unit according to any of the preceding items [00167]-[00168], configured such that the colour temperature of the visible polychromatic light is between 2500 K and 5000 K, such as around 4500 K, preferably around 2700 K.

The light emitting unit according to any one of the preceding items [00167]-[00169], configured for emittance of the polychromatic visible light for a first predefined period of time of less than 16 hours per day, and configured for emittance of the monochromatic non-visible UV-B and UV-C light, for a second predefined period time of at least 22 hours per day.

The light emitting unit according to any of the preceding items, wherein all LEDs are mounted on a common replaceable circuit board.

The light emitting unit according to any of the preceding items, comprising at least one light sensor for measuring the light exposure from the light emitting unit.

The light emitting unit according to any of the preceding items, comprising at least one movement sensor for detecting activity, e.g. movement from humans and/or animals, in the vicinity of the light emitting unit.

The light emitting unit according to any of the preceding items, configured to turn off the UV-B LED(s) and/or the UV-C LED(s) when activity is detected in vicinity of the light emitting unit.

The light emitting unit according to any one of the preceding items, comprising a housing, preferably metallic, preferably having cooling fins, the housing accommodating all LEDs of the light emitting unit.

The light emitting unit according to any one of the preceding items [00167]-[00175], comprising a single LED having a wattage of least 48 W for providing the polychromatic visible light, and one or more LEDs having wattages of 1 W, 3 W, 12 W, 48 or 100 W, for providing each of the UV-B light, optionally the UV-C light.

A system for 1) providing working light, 2) reducing the microbial pressure, and/or 3) stimulating the production of natural vitamin D3, in a room for accommodating humans, such as a classroom, a hospital ward, an office space, or an assembly hall, or in an animal farm production facility, the system comprising:
at least one of the light emitting units according to any of the preceding items, and
a control system adapted for managing
a. light exposure time, and/or
b. light exposure intensity,
i. in selected wavelength ranges of the light emitting unit(s).

The system of item [00177], comprising at least one light sensor for measuring the light exposure from the light emitting unit(s).

The system according to any of preceding items [00177]-[00180], comprising at least one movement sensor for detecting activity in the room.

The system according to any of preceding items [00177]-[00181], configured for maintaining emittance of a predefined light exposure intensity in selected wavelength ranges of the light emitting unit(s) based on measurements of the light exposure from the light emitting unit(s).

The system according to any of preceding items [00177]-[00182], configured such that the light emitting units only emit UV-C light during a selected period during the day, such as during closing time of the room, such as night-time, for example from 22 to 5 o'clock local time.

The system according to any of preceding items [00177]-[00183], configured such that the light emitting units only emit UV-C light when humans and/or animals are not present in vicinity of the light emitting unit(s), such as in a corresponding room containing the light emitting system.

The system according to any of preceding items [00177]-[00184], configured to control a ratio of light emittance, such as based on input from sensor(s), between the at least second UV-B LEDs and the at least first UV-B LED(s) such that the ratio of total light emittance of 283±5 nm light relative to total light emittance of 297±5 nm light can be selected.

The system according to any of preceding items [00177]-[00185], configured to control a ratio of light emittance, such as based on input from sensor(s), between the at least first UV-B LEDs and the at least first UV-C LED(s) such that the ratio of total light emittance of 297±5 nm light relative to total light emittance of 233±5 nm light can be selected.

1. A light emitting unit for 1) reducing microbial pressure, and 2) stimulating production of natural vitamin D3, the light emitting unit comprising:
at least a first UV-B LED configured for emitting monochromatic UV-B light having a maximum intensity between 292-302 nm, more preferably between 295-299 nm, most preferably at 297 nm;
and
at least a second UV-B LED configured for emitting monochromatic UV-B light having a maximum intensity between 275-290 nm, preferably between 278-288 nm, more preferably between 281-285 nm, most preferably at 283 nm;
and/or
at least a first UV-C LED configured for emitting monochromatic UV-C light having a maximum intensity between 228-238 nm, preferably between 231-235 nm, most preferably at 233 nm.

2. The light emitting unit according to claim 1, wherein at least the first and the second monochromatic UV-B LED light have a full width half max (FWHM) spectral bandwidth of less than less than or equal to 20 nm, more preferably less than or equal to 15 nm, most preferably less than or equal to 10 nm or even 8 nm.

3. The light emitting unit according to claim 1, wherein the at least second monochromatic UV-B LED light has a full width half max (FWHM) spectral bandwidth of at least 30 nm, more preferably at least 35 nm, most preferably at least 40 nm.

4. The light emitting unit according to claim 1, wherein the at least first UV-B LED is configured for emitting monochromatic UV-B light having a maximum intensity between 296-298 nm, most preferably at 297 nm.

5. The light emitting unit according to claim 1, wherein the at least second UV-B LED is configured for emitting monochromatic UV-B light having a maximum intensity between 282-284 nm, most preferably at 283 nm.

6. The light emitting unit according to claim 1, wherein the at least first UV-C LED is configured for emitting monochromatic UV-C light having a maximum intensity between 232-234 nm, most preferably at 233 nm.

7. The light emitting unit according to claim 1, configured to control a ratio of light emittance between the at least first UV-B LED and the at least second UV-B LED.

8. The light emitting unit according to claim 1, wherein a ratio of light emittance between the at least second UV-B LED and the at least first UV-B LED is more than 2, more preferably more than 2.5, most preferably around or more than 3.

9. The light emitting unit according to claim 1, further comprising:
at least a second UV-C LED configured for emitting monochromatic UV-C light having a maximum intensity between 217-227 nm, preferably between 220-224 nm, most preferably at 222 nm.

10. The light emitting unit according to claim 1, further comprising:
at least a third UV-C LED configured for emitting monochromatic UV-C light having a maximum intensity between 255-265 nm, preferably between 258-262 nm, most preferably at 260 nm.

11. The light emitting unit according to claim 1, wherein the monochromatic UV-C LED light has a full width half max (FWHM) spectral bandwidth of less than or equal to 20 nm, more preferably less than or equal to 15 nm, most preferably less than or equal to 10 nm or even 8 nm.

12. The light emitting unit according to claim 1, configured to control a ratio of light emittance between at least one of the UV-B LED and the at least one UV-C LED.

13. The light emitting unit according to claim 1, comprising at least one visible light LED configured for emitting polychromatic visible light, preferably with wavelengths in the range of 380 nm-750 nm.

14. The light emitting unit according to claim 1, configured for not emitting light below 270 nm, except emittance of monochromatic UV-C light in a range of 215-240 nm.

15. The light emitting unit according to claim 13, configured such that a colour temperature of the visible polychromatic light is around 2700 K.

16. The light emitting unit according to claim 13, configured for emittance of the polychromatic visible light for a first predefined period of time of less than 16 hours per day, and configured for emittance of the monochromatic non-visible UV-B and UV-C light, for a second predefined period time of at least 22 hours per day.

17. The light emitting unit according to claim 1, wherein all of the LEDs are mounted on a common replaceable circuit board.

18. The light emitting unit according to claim 1, further comprising at least one light sensor for measuring light exposure from the light emitting unit.

19. The light emitting unit according to claim 1, further comprising at least one movement sensor for detecting activity, e.g. movement from humans and/or animals, in a vicinity of the light emitting unit.

20. The light emitting unit according to claim 19, configured to turn off the UV-B LED(s) and/or the UV-C LED(s) when activity is detected in the vicinity of the light emitting unit.

21. The light emitting unit according to claim 1, further comprising a housing, preferably metallic, preferably having cooling fins, the housing accommodating all LEDs of the light emitting unit.

22. The light emitting unit according to claim 13, comprising a single LED having a wattage of least 48 W for providing the polychromatic visible light, and one or more LEDs having wattages of 1 W, 3 W, 12 W, 48 or 100 W, for providing each of the UV-B light, optionally the UV-C light.

23. A system for 1) providing working light, 2) reducing microbial pressure, and/or 3) stimulating production of natural vitamin D3, in a room for accommodating humans, such as a classroom, a hospital ward, an office space, or an assembly hall, or in an animal farm production facility, the system comprising:
at least one of the light emitting units according to claim 1;
and
a control system adapted for managing;
light exposure time; and/or
light exposure intensity, such as total light emittance;
in selected wavelength ranges of the light emitting unit(s).

24. The system of claim 23, further comprising at least one light sensor for measuring the light exposure from the light emitting unit(s).

25. The system according to claim 23, further comprising at least one movement sensor for detecting activity in the room.

26. The system according to claim 23, configured for maintaining emittance of a predefined light exposure intensity in selected wavelength ranges of the light emitting unit(s) based on measurements of the light exposure from the light emitting unit(s).

27. The system according to claim 23, configured such that the light emitting units only emit UV-C light during a selected period during the day, such as during closing time of the room, such as night-time, for example from 22 to 5 o'clock local time.

28. The system according to claim 23, configured such that the light emitting units only emit UV-C light when humans and/or animals are not present in vicinity of the light emitting unit(s), such as in a corresponding room containing the light emitting system.

29. The system according to claim 23, configured to control a ratio of light emittance, such as based on input from sensor(s), between the at least first UV-B LEDs and the at least second UV-B LED(s) such that the ratio of total light emittance of 283±5 nm light relative to total light emittance of 297±5 nm light can be selected.

30. The system according to claim 23, configured to control a ratio of light emittance, such as based on input from sensor(s), between the at least first UV-B LEDs and the at least first UV-C LED(s) such that the ratio of total light emittance of 297±5 nm light relative to total light emittance of 233±5 nm light can be selected.
